(19) United States
(12) Patent Application Publication
(10) Pub. No.: US 2012/0172576 A1
(43) Pub. Date: Jul. 5, 2012

(54) METHOD AND MARKER FOR SIMPLE TRANSFORMATION AND SELECTION OF RECOMBINANT PROTISTS

(76) Inventor: Matthias Rüsing, Koln (DE)

(21) Appl. No.: 13/339,454

(22) Filed: Dec. 29, 2011

Related U.S. Application Data
(62) Division of application No. 10/395,435, filed on Mar. 24, 2003, now abandoned.

(30) Foreign Application Priority Data
Mar. 30, 2002 (DE) 102 14 406.0

(51) Int. Cl. C07K 14/00; C12N 1/11; C12P 21/06; C12N 15/74

(52) U.S. Cl. 530/350; 435/471; 435/258.1; 435/69.1

(57) ABSTRACT

The present invention concerns a method for production of genetically modified (recombinant) protists without using negative selection markers, in which an auxotrophic mutant of the protist is produced, this mutant is then transformed with recombinant DNA containing at least one gene for complementation of the corresponding auxotrophy, and the resulting recombinant protist is finally selected on a minimal medium that makes possible growth of only the correspondingly complemented protist. The present invention also concerns an efficient method for production of proteins by protists so modified, in which the gene for the protein being produced is coupled to the marker gene.

The present invention the method for production of genetically modified (recombinant) protists without the use of negative selection markers, and an efficiency method for production of proteins by such modified protists.

Production of recombinant proteins by heterologous protein expression represents an alternative to recovery of proteins from natural sources. Natural sources for proteins, which serve as pharmaceuticals, for example, are often limited, very expensive to purify or simply not available. They can also be very problematical, because of the hazard of toxic or especially infectious contaminants. On the other hand, biotechnology now permits economical and safe production of an entire series of proteins in sufficient amounts by heterologous expression for a wide variety of applications: for example, antibodies (for diagnosis, passive immunization and research), hormones (like insulin, erythropoietin (EPO), interleukins, etc. for therapeutic use), enzymes (for example, for use in food technology, diagnosis, research), blood factors (for treatment of hemophilia), vaccines, etc. (Glick & Pasternak 1998, Molecular Biotechnology, ASM Press, Washington, D.C., Chapter 10: 227-252).

Proteins for medical use, especially human proteins, must be identical in biochemical, biophysical and functional properties to the natural protein. During recombinant production of such proteins by heterologous gene expression, it is therefore kept in mind that a number of f post-translational protein modifications are present in eukaryotic cells in contrast to bacteria: formation of disulfide bridges, proteolytic cleavage of precursor proteins, modifications of amino acid residues (phosphorylation, acetylation, acylation, sulfatization, carboxylation, myristylation, palmitylation, and especially glycosylations). Moreover, proteins in eukaryotic cells are only brought to the correct three-dimensional structure by a complex mechanism with participation of chaperones.

These modifications play a very important role with respect to specific structural and functional properties of proteins, like activity of enzymes, specificity (receptor binding, cell recognition), folding, solubility, etc. of the proteins (Ashford & Platt 1998, in: Post-translational Processing—A Practical Approach Ed. Higgins & Hames, Oxford University Press, Chapter 4: 135-174; Glick & Pasternak 1998, Molecular Biotechnology, ASM Press, Washington, D.C., Chapter 7: 145-169).

Modifications deviating from the natural structure can lead to inactivation of the proteins or possess high allergenic potential.

Although an entire series of bacterial and eukaryotic expression systems are established for production of recombinant proteins, there is no universal system that covers the entire spectrum of possible protein modifications, especially in eukaryotic proteins, and would therefore be universally employable (Castillo 1995, Bioprocess Technology 21: 13-45, Geise et al. 1996, Prot. Expr. Purif. 8: 271-282; Verma et al. 1998 J. Immunological Methods 216: 165-181; Glick & Pasternak 1998, Molecular Biotechnology ASM Press, Washington, D.C., Chapter 7: 145-169). Another problem is that some of the frequently used systems introduce unusual and sometimes undesired post-translational protein modifications. Recombinant expressed proteins from yeasts are sometimes modified extremely strongly with mannose residues. These so-called “high-mannose” structures form yeast consist of about 8-50 mannose residues and therefore differ significantly from the mannose-rich glycoprotein structures from mammal cells, which have a maximum of 5-9 mannose residues (Moreman et al. 1994, Glycobiology 4(2): 113-125). These yeast-typical mannose structures are strong allergens and therefore problematical in production of recombinant glycoproteins for therapeutic use (Tuite et al. 1999, in Protein Expression—A Practical Approach, Ed. Higgins & Hames, Oxford University Press, Chapter 3: especially page 76). In addition, no hybrid or complex glycoprotein structures can be formed in yeasts, which further constrains their use as an expression system.

Plants that have recently been discussed and used increasingly more often as production systems for recombinant proteins, on the other hand, have xyloses on the glycoprotein structures, instead of the sialic acid typical of mammals (Ashford & Platt, see above). Xyloses and the α-1,3-linked fucoses also detected in plants can represent an allergic risk and are therefore also problematical (Jenkins et al. 1996, Nature Biotech. 14: 975-981).

A large demand therefore exists precisely for new eukaryotic expression systems, primarily as an alternative to the very cost-intensive and demanding production of recombinant proteins with mammal culture cells.

Such a system would ideally meet the following requirements: 1) Selection markers and regulative DNA elements (like transcription and translation signals, etc.) must be available. 2) The expression system should make possible important eukaryotic post-translational protein modifications, but not produce allergens for humans, and 3) production of recombinant proteins should be as simple and economical as possible, for example, by the possibility of fermentation of the cells or organisms on a production scale (for example, several thousand L) on simple media and simple workup of the products.

Protozoans or protists (for definition, see Henderson's Dictionary of Biological Terms, 10^{th} Edition 1989, Eleanor Lawrence, Longman Scientific & Technical, England or Margulis et al. (Editors) 1990. Handbook of Protoctista, Jones & Bartlett, Boston; van den Hoek et al. 1995, Algae—An Introduction to Phycology, Cambridge University Press) might represent an interesting alternative to the already established eukaryotic expression systems, like yeast, mammal or insect culture cells. These organisms are a very heterogeneous group of eukaryotic, generally unicellular microorganisms. They possess the compartmentalization and differentiation typical of eukaryotic cells. Some are relatively closely related to higher eukaryotes, but, on the other hand, are more similar to yeasts or even bacteria with respect to culturing and growth and can be fermented relatively easily at high cell density on simple media on a large scale.

An interesting protist for expression of heterologous proteins is the ciliate Tetrahymena, especially Tetrahymena thermophila. This is a nonpathogenic, unicellular, eukaryotic microorganism that is relatively closely related to the higher eukaryotes and has the cell differentiations typical of them. The post-translational protein modifications in Tetrahymena are more strongly similar to those in mammal cells than those detected in yeast or other eukaryotic expression systems. For example, no strongly antigenic sugar chains are found in Tetrahymena on the glycoproteins, as in yeasts (“high mannose” structures) and expression systems based on plant or lower animal cell cultures (xylose residues) (see above). Although Tetrahymena is a true, complexly differentiated eukaryote, it is similar in its culturing and growth properties to the simple yeasts or bacteria and can be fermented well on relatively inexpensive skim milk media on a large scale. The generation time under optimal conditions is about 1.5-3 h and very high cell densities (2.2×10⁷ cell/mL, corresponding at 48 g/L of dry weight) can be reached (Kiy and Tiedke 1992, Appl. Microbiol. Biotechnol. 37: 576-579; Kiy and Tiedke 1992, Appl. Microbiol. Biotechnol. 38: 141-146). Tetrahymena is consequently very interesting for fermentative production of recombinant proteins on a large scale.

Another advantageous aspect of Tetrahymena as an expression system is the fact that integration of the heterologous gene by homologous DNA recombination is possible in Tetrahymena. Because of this, mitotically stable transformants can be generated. Targeted gene “knockouts” are also possible by homologous DNA recombination (Bruns & Cassidy-Hanley in: Methods in Cell Biology, Volume 62, Ed. Asai & Formey, Academic Press (1999) 501-512); Hai et al. in: Methods in Cell Biology, Volume 62, Ed. Asai & Formey, Academic Press (1999) 514-531; Gaertig et al. (1999) Nature Biotech. 17: 462-465 or Cassidy-Hanley et al. 1997 Genetics 146: 135-147). In addition, the somatic macronucleus or the generative micronucleus can be alternately transformed. During macronucleus transformation, sterile transformants are obtained, which can be advantageous relative to safety or acceptance questions.

Transformation of Tetrahymena can be achieved by microinjection, electroporation or microparticle bombardment. A number of vectors, promoters, etc. are available for this. Selection of the transformants occurs by a resistance marker. Thus, Tetrahymena was successfully transformed with an rDNA vector. Selection occurred in this case with a paromomycin-resistance mutation of rRNA (Tondravi et al. 1986, PNAS 83:4396; Yu et al. 1989, PNAS 86: 8487-8491). In other transformation experiments, cycloheximide or neomycin resistance were successfully expressed in Tetrahymena (Yao et al. 1991, PNAS 88:9493-9497; Kahn et al. 1993, PNAS 90: 9295-9299). In addition to these marker genes, Gaertig et al. (1999, Nature Biotech. 17: 462-465) successfully expressed two recombinant proteins in Tetrahymena (a fish parasite antigen and partial ovalbumin from the chicken). Selection occurred with Paclitaxel (Taxol). This system developed by Gaertig et al. has a patent pending (WO 00/46381).

Methods for transformation and heterologous protein expression have only been described for a few protists or protozoans. Paramecium can be mentioned here as another cilitate (Boileau et al. 1999, J. Eukaryot. Microbiol. 46: 55-65). Various experiments on transformation and expression of recombinant proteins, however, were also carried out in parasitic protozoans, like Trypanosoma, Leishmania, Plasmodium and others (Beverly 2000, WO 00/58483). A review is provided by Kelly (1997, Advances in Parasitology, Vol. 39, 227-270). One possibility for heterologous protein expression was also demonstrated in the slime mold Dictyostelium discoideum (Manstein et al. 1995, Gene 162: 129-134, Jung and Williams 1997, Biotechnol. Appl. Biochem. 25: 3-8), but also in photoautotrophic protists (microalgae), like Chlamydomonas (Hall et al. 1993, Gene 124: 75-81), Volvox (Schiedlmeier et al. 1994, PNAS 91: 5080-5084), certain dinoflagellates (ten Lohuis & Miller 1998, Plant Journal 13: 427-435) and diatomes (Dunahay et al. 1995, J. Phycol. 31: 1004-1012). In most cases, however only simple resistance marker or non-human selection markers were expressed.

None of these organisms has thus far been used on a larger scale for production of recombinant proteins. A major problem for many of these and other possibly interesting systems is the absence of well established genetic engineering methods, and especially molecular biological “tools”, like vectors, markers, etc.

The presence of a selective marker is a necessary condition, in order to be able to deliberately modify cells genetically. Selection of transformed cells or organisms generally occurs through negative selection markers, generally resistance to an antibiotic (for example, ampicillin, kanamycin, tetracycline, neomycin, etc.). Selection rarely occurs by incorporating an essential gene into a defective cell type (in terms of this gene product) (positive selection or selection by complementation). These include, for example, LEU- or URA3-based selection systems in yeasts (see Glick & Pasternak, Molecular Biotechnology, Principles and Applications of Recombinant DNA, 1998, 2^{nd} Edition, ASM Press, Washington D.C., pages 109-169). For the still poorly investigated “new organisms”, like the already mentioned protists, however, this second approach is not available. The usefulness of this system, especially for production of recombinant proteins on a production scale, is therefore strongly restricted.

Negative selection generally has serious shortcomings. In the first place, DNA unnecessary for the desired product must be introduced to the production organism, which can raise objections in terms of biological safety, but especially public acceptance. On the other hand, the organisms must be cultured in the presence of the corresponding antibiotic during the entire production time to maintain selection pressure, which enormously drives up the costs. Not only must the costs for the antibiotic itself be considered, along with disposal of production waste (media, etc.), but workup of the proteins can prove to be much more difficult. However, apart from economic considerations, environmental compatibility, biological and genetic engineering safety, and especially public acceptance, are classified as very problematical. In addition, problems of a purely technical nature are posed if the organism must be repeatedly transformed. The simultaneous presence of several antibiotics with simultaneous expression of several antibiotic resistance genes can have an extremely adverse effect on the organisms, if it is possible at all, or lead to unforeseen side effects.

In view of the prior art, the task of the present invention was to provide a method for positive selection of genetically modified protists that permits efficient production or recombinant proteins, among other things.

This is solved, along with additional, not explicitly mentioned tasks that can be easily derived or concluded from the context just discussed by the versions of the present invention defined in the patent claims.

A method for production of recombinant protists can be made available in surprisingly simple fashion by producing an auxotrophic mutant of protists, transforming these mutants with recombinant DNA containing at least one gene for complementation of the corresponding auxotrophy and finally selecting the resulting recombinant protist on a minimal medium that permits growth only for the complemented protists. In particular, in order to permanently maintain selection pressure, neither selection for a resistance to an antibiotic nor the presence of undesired and possibly heterologous genes in the recombinant organism is ultimately necessary for this method. Addition of antibiotics to the culture medium can therefore be dispensed with. The method can also be used without problem repeatedly on the same organism strain, transforming it with a variety of desired recombinant genes without any (over) expression of additional or undesired genes.

Under another aspect of the present invention, a method for production of recombinant proteins can be made available also in simple fashion by producing recombinant protists in the manner just described, in which the recombinant DNA additionally contains at least one functional recombinant gene for a protein being expressed for transformation of the protists. The recombinant protists are then cultured so that the proteins are expressed and can then be isolated.

Another aspect of the present invention concerns a recombinant protists, characterized by the fact that it contains a mutation that knocks out an essential gene, in which the resulting auxotrophy is preferably complemented by transformation of the protists with recombinant DNA.

In a preferred variant of the present invention, production of the auxotrophic mutants occurs by knockout of an essential gene. Knockout can be achieved by complete deletion of the corresponding gene or by its mutation. Mutations are understood to mean, for example, insertions, deletions, inversions or merely exchange of individual base pairs. Gene deletions or mutations could be introduced to the target organism by methods known to one skilled in the art. Among other things, in vitro mutagenesis works here, for example, by error-prone PCR, or perhaps according to the clinical method (Sambrook et al., Molecular Cloning, A Laboratory Manual, Coldspring Harbor, N.Y.), or exon shuffling or gene site saturation mutagenization (GSSM) (see, for example: www.diversa.com and www.maxygen.com).

For the purposes of the present invention, essential genes for production of an auxotrophic knockout mutant are understood to mean essential metabolic genes, for example, for fatty acid, sterol, amino acid biosynthesis, etc., whose elimination can be compensated by adding the corresponding molecules (fatty acids, sterols, amino acids, etc.) to the culture medium (then also called markers). By deliberate knockout of such genes, the cells become auxotrophic for products of this metabolic pathway. In the present example, this is described, for example, for sterol and fatty acid biosynthesis. By addition of the corresponding metabolic product, i.e., cholesterol or fatty acids, the cells can survive this knockout. Without these additions, the cells quickly die.

Genes that code, for example, for a triterpenoid-cyclase (synonym for tetrahymanol-cyclase), a delta-6-desaturase or a delta-9-desaturase, are therefore appropriate targets according to the invention for knockout, in order to produce an auxotrophic mutant of the respective organism.

A preferred gene according to the invention for a triterpenoid-cyclase was described in German Patent Application DE 199 57 889 A1. A preferred gene according to the invention for a delta-6-desaturase was described in German Patent Application DE 100 44 468 A1. A preferred gene according to the invention for a delta-9-desaturase was described by Nakashima S. et al. in Biochem. J. 317, 29-34 (1996), and a respective sequence can be found in GenBank under accession no.: EMBL D83478. Concerning GenBank, see Benson, D. A. et al., Nuc. Acid Res., 28 (10), 15-18 (2000). All these references are incorporated in their entirety in the present application.

The cells according to the invention reacquire their auxotrophy for the metabolic product by reincorporation of the knockout gene. In this case, one says that auxotrophy is complemented. Selection for successfully transformed cells occurs in minimal medium, i.e., especially omitting the metabolic product, for which the unsuccessfully transformed organisms are auxotrophic. Minimal medium according to the invention is understood to mean a medium containing all the necessary building blocks that permit survival of the cells (carbon sources, like sugar, nitrogen sources, possibly amino acids, vitamins, trace elements, etc.), but do not contain the metabolic product for which the initial organism is auxotrophic.

If this gene that complements auxotrophy is now coupled to a gene that codes for a heterologous protein being expressed, the transformants can be identified without addition of a selection marker and successful stable expression is also not dependent on addition of a selection marker, as is typically the case, for example, in recombinant E. coli. As an additional positive effect, no foreign DNA is introduced to the cell, in addition to the target gene being expressed. In addition, in organisms in which homologous recombination occurs (for example, Tetrahymena), the DNA is not randomly integrated in the genome, but at a specific position, namely, the natural position of the marker gene.

However, it is clear to one skilled in the art that complementation of auxotrophy can also occur by corresponding heterologous or in vitro modified genes.

According to the invention, transformed protists, protozoans are suitable for selection according to the process just described, for example, ciliates, preferably of the genera Paramecium or Tetrahymena, especially the species Tetrahymena thermophila.

Recombinant DNA for transformation of auxotrophic protist mutants can be a vector, for example, i.e., any type of nucleic acid, like a plasmid, cosmid, virus, an autonomously replicating sequence, a phage, a linear or circular, single- or double-strand DNA or RNA molecule that can replicate in the target organism itself or be incorporated into its genome, but at least contains functional sequences in the target organism.

Functional sequences according to the invention are understood to mean those DNA sections that can meet their corresponding function even in the recombinant organism.

A functional gene is understood for the purposes of the present invention to mean a gene that can be expressed in the target organism. In particular, a functional gene therefore includes, in addition to a coding sequence, a promoter functional in the target organism that leads to transcription of the coding sequence. A functional protein of this type can have, among other things, one or more TATA boxes, CCAAT boxes, GC boxes or enhancer sequences. In addition, the functional gene can include a functional terminator in the target organism that leads to interruption of transcription and contains signal sequences that lead to polyadenylation of mRNA. The coding sequence of the functional gene also has all the properties necessary for translation of the target organism (for example, start codon (for example, ATG), stop codon (for example, TGA, especially in Tetrahymena), A-rich regions before the start (translation initiation sites), Kozak sequences, poly-A site. The gene can also have the specific codon usage for the corresponding recombinant organism (for Tetrahymena, see, for example, Wuitschick & Karrer, J. Eukaryot. Microbiol. (1999)).

According to the invention, the recombinant gene for a protein to be expressed in a recombinant protist in a method for production of recombinant proteins is a homologous or heterologous gene. If a heterologous gene is involved, it is preferably isolated from vertebrates, especially from humans. A preferred example of this is human erythropoietin. Other preferred recombinant genes according to the invention for proteins to be expressed in recombinant protists are those from organisms that can trigger diseases in man or animals (for example: malaria), in order to be able to achieve active immunization by means of a recombinant protein, or also biomass or parts of it, containing the recombinant proteins.

### DESCRIPTION OF THE FIGURES

FIG. 1: Structure of tetrahymanol gene pgTHC

FIG. 2: tetrahymanol knockout construct pgTHC::neo

FIG. 3: Expression construct pBTHC

FIG. 4: Structure of tetrahymanol cyclase/neo construct pgTHC+neo

FIG. 5: Delta-6-desaturase knockout construct pgDES6::neo

FIG. 6: Structure of the genomic delta-6-desaturase gene pgDES6

FIG. 7: Structure of the delta-6-desaturase/neo construct pgDES6+neo

The following examples serve to explain the invention without restricting the invention to these examples.

### EXAMPLE 1

### Organisms and Culturing Conditions

Tetrahymena thermophila (strains B1868 VII, B208611, B*VI, CU428, CU427, CU55, furnished by Dr. J. Gaertig, University of Georgia, Athens, Ga., USA) were cultured in modified SPP medium (2% proteose peptone, 0.1% yeast extract, 0.2% glucose, 0.003% Fe-EDTA (Gaertig et al. (1994) PNAS 91:4549-4553)) and skim milk medium (2% skim milk powder, 0.5% yeast extract, 1% glucose, 0.003% Fe-EDTA) or MYG medium (2% skim milk powder, 0.1% yeast extract, 0.2% glucose, 0.003% Fe-EDTA) with addition of antibiotic solution (100 U/mL penicillin, 100 μg/mL streptomycin and 0.25 μg/mL amphotericin B (SPPA medium) at 30° C. in 50 mL volumes in 250 mL Erlenmeyer flasks during shaking (150 rpm).

Plasmids and phageas were multiplied and selected in E. coli XL1-Blue MRF′, TOP10F′ or J109 (Stratagene, Invitrogen, GibcoBRL, Life Technologies) culturing of the bacteria occurred under standard conditions in LB or NZY medium with antibiotics in standard concentrations (Sambrook et al. (1989) Molecular Cloning: A Laboratory Manual, Cold Spring Harbor Laboratory, Cold Spring, N.Y.).

### EXAMPLE 2

### Production of Triterpenoid-Cyclase Knockout Construct

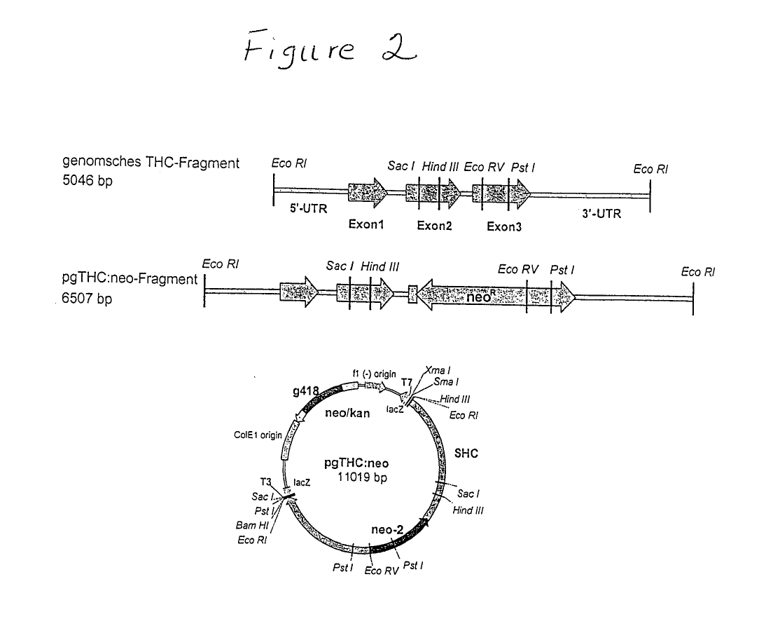

To produce the knockout constructs, a neo-cassette from the plasmid p4T2-1ΔH3 (Gaertig et al. (1994) Nucl. Acids Res. 22: 5391-5398) was inserted into the genomic sequence of triterpenoid-cyclase (Patent Application DE 199 57 889 A1). This is a neomycin resistance gene under the control of the Tetrahymena histon H4-promoter and the 3′ flanking sequence of the BTU2 gene. This construct in Tetrahymena mediates resistance to paromomycin. The plasmid p4T2-1ΔH3 was cleaved with Eco RV/Sma I and the roughly 1.4 kb fragment, including the neo-cassette, was ligated into the genomic sequence of Tetrahymena triterpenoid-cyclase with plasmid pgTHC cleaved with Eco RV. Because of this, the plasmid pgTHC::neo is produced (see FIG. 2). During a successful transformation, the gene for triterpenoid-cyclase was replaced by this construct by homologous recombination, so that resistance of the cells to paromomycin was mediated.

### EXAMPLE 3

### Production of the Expression Construct pBTHC

The vector pBICH3 (Gaertig et al. 1999 Nature Biotech. 17: 462-465, WO 00/46381) contains the coding sequence of the Ichthyophthirius I antigen (G1) preprotein, flanked by the non-coding, regulatory sequences of Tetrahymena thermophila BTU1 gene. A modified plasmid (pBICH3-Nsi) with an NSi I cleavage site at the start (made available by J. Gaertig, University of Georgia, Athens, Ga., USA) was used, in order to produce the tetrahymanol-cyclase expression construct pBTHC. For this purpose, the tetrahymanol-cyclase of Tetrahymena was inserted by PCR Nsi I and Bam HI cleavage sites at the start and stop of the coding sequences. Isolated plasmids that contain the complete cDNA sequences of tetrahymanol-cyclase (pTHC) were used as template for PCR. The primers

(SEQ ID no. 1)
THC-Nsi-F:
5′-CTCTTTCATACATGCATAAGATACTCATAGGC-3′
and
(SEQ ID no. 2)
THC-Bam-R:
5′-GGCTTGGATCCTCAAATATTTTATTTTTATACAGG-3′

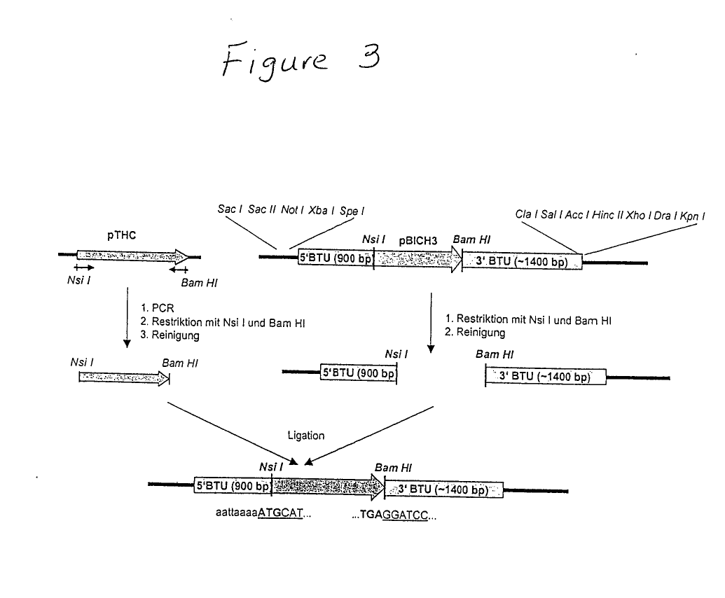

produced PCR products that contained the complete coding sequence of tetrahymanol-cyclase, flanked by Nsi I and Bam HI cleavage sites. The PCR products and plasmid pBICH3-Nsi were cleaved with the restriction enzymes Nsi I and Bam HI, purified with agarose gel and ligated (see FIG. 3). The expression construct pBTHC so produced contained the complete sequence coding for the triterpenoid-cyclase inserted in a correct reading frame in the regulatory sequences of the BTU1 gene. The constructs were linearized for transformation of Tetrahymena by digestion with the restriction enzymes Xba I and Sal I.

During a successful transformation, the BTU1 gene was replaced by this construct by homologous recombination, so that resistance of the cells to Paclitaxel was mediated.

### EXAMPLE 4

### Macronucleus Transformation of Tetrahymena with Tetrahymanol-Cyclase Expression Construct pBTHC

5×10⁶ Tetrahymena thermophila cells (CU522) were used for a transformation. Culturing of cells occurred in 50 mL SPPA medium at 30° C. in a 250 mL Erlenmeyer flask on a rocking device of 150 rpm to a cell density of about 3−5×10⁵ cells/mL. The cells were pelletized for 5 minutes by centrifuging (1200 g) and the cell pellet was resuspended in 50 mL 10 mM tris-HCl (pH 7.5) and centrifuged as before. This washing step was repeated and the cells resuspended in 10 mM tris-HCl (pH 7.5 plus antibiotic) at a cell density of 3×10⁵ cell/mL, transferred to a 250 mL Erlenmeyer flask and incubated for 16-20 hours without shaking at 30° C. (hunger phase). After the hunger phase, the cell count was determined again, centrifuged as above and the cells were set at a concentration of 5×10⁵ cell/mL with 10 mM tris-HCl (pH 7.5). 1 mL of the cell suspension was used for the transformation. The transformation occurred by microparticle bombardment (see below). For regeneration, the cells were taken up in SSPA medium and incubated at 30° C. without shaking in the Erlenmeyer flask. After 3 hours, Paclitaxel® was added in a final concentration of 20 μm and the cells transferred in 100 μL aliquots to 96-well microtiter plates. The cells were incubated in a moist, darkened box at 30° C. After 2-3 days, Paclitaxel-resistant clones could be identified. Positive clones were reinoculated in fresh medium with 25 μm Paclitaxel. By culturing of the cells in increasing Paclitaxel concentration (to 80 μm), a complete “phenotypic assortment” was reached (Gaertig & Kapler (1999)).

For analysis of the clones, about 4 mL cultures in SPPA were cultured with Paclitaxel, the DNA isolated (Jacek Gaertig et al. (1994) PNAS 91: 4549-4553) and DNA integrated in the BTU1 locus, amplified by PCR. The BTU1-specific primer BTU1-5′F (AAAAATAAAAAAGTTTGAAAAAAAACCTTC (SEQ ID no. 3)) served as primer, about 50 bp before the start codon and BTU1-3R′ (GTTTAGCTGACCGATTCAGTTC (SEQ ID no. 4)), 3 bp behind the stop codon. The PCR products were analyzed uncleaved and cleaved with Hind III, Sac I or Pst I on 1% agarose gel. The complete “phenotypic assortment” was checked via RT-PCR with the BTU1-specific primers (Gaertig & Kapler (1999)).

### EXAMPLE 5

### Production of the Delta-6-Desaturase Knockout Construct pgDES6::neo

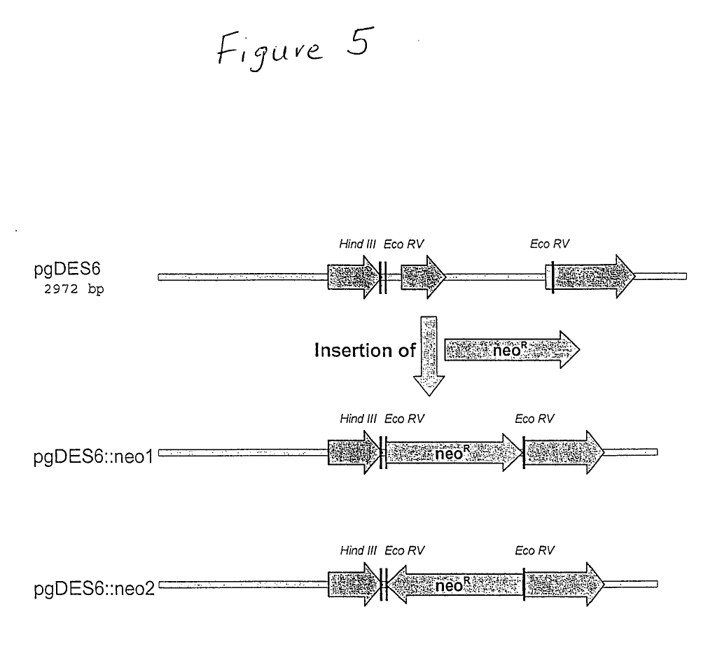

For production of the knockout construct, a neo-cassette from the plasmid p4T2-1ΔH3 (Patent Application DE 100 44 468 A1) was inserted into the genomic sequence of delta-6-desaturase. This is a neomycin resistance gene under the control of the Tetrahymena histon H4-promoter and the 3′ flanking sequence of the BTU2 gene. This construct in Tetrahymena mediates resistance to paromomycin. The plasmid p4T2-1ΔH3 was cleaved with Eco RV/Sma I and the roughly 1.4 kb fragment of the neo-cassette was ligated into the genomic sequence of Tetrahymena delta-6-desaturase (see FIG. 5) with the plasmid pgDES6 (Patent Application DE 100 44 461 A1) cleaved with Eco RV. The plasmid pgDES6::neo was produced. During a successful transformation, the gene for Delta-6-desaturase was replaced by this construct by homologous recombination, so that resistance of the cells to paromomycin was mediated.

### EXAMPLE 6

### Micronucleus and Macronucleus Transformation of Tetrahymena with the Knockout Constructs pgTHC::neo and pgDES6::neo

Tetrahymena strains of different pairing type (CU428 VII and B2086 II) were cultured separately in SPPA medium at 30° C. during shaking (150 rpm) in Erlenmeyer flasks. At a cell density of 3−5×10⁵ cells/mL, the cells were centrifuged for 5 minutes at room temperature (1200 g). The cells were washed three times with 50 mL 10 mM tris-HCl (pH 7.5) and finally resuspended in 50 mL 10 mM tris-HCl (pH 7.5) and mixed with antibiotic solution, and then incubated without shaking in an Erlenmeyer flask at 30° C. After about 4 hours, the cell count of both cultures was determined again and set at 3×10⁵ cells/mL with 10 mM tris-HCl (pH 7.5). The cultures were then incubated for another 16-20 hours at 30° C. After this hunger phase, the same (absolute) cell count was mixed from both cultures in a 2 L Erlenmeyer flask. The cells were incubated at 30° C. (beginning of conjugation) and the efficiency of conjugation was determined after 2 hours. For a successful transformation, about 30% of the cells had to be present at this point as pairs.

For micronucleus transformation, 1×10⁷ conjugating cells (5×10⁶ pairs), 3 hours, 3.5 hours, 4 hours and 4.5 hours after the beginning of conjugation, were centrifuged for 5 minutes at 1200 g and the cell pellet resuspended in 1 mL 10 mM tris-HCl (pH 7.5).

For transformation of the new macronucleus charges, the cells, 11 hours after the beginning of conjugated, were centrifuged as above and resuspended in tris-HCl.

Transformation occurred by microparticle bombardment (see below).

For culturing of the tetrahymanol-cyclase knockout mutants, 10 μg/mL cholesterol was added to the medium.

For culturing of the delta-6-desaturase knockout mutants, 200 μg/mL Borage oil (20-25% GLA; SIGMA) was added to the medium.

Transformed cells could be identified by selection for paromomycin resistance. During transformation of the micronucleus, 11 hours after the beginning of conjugation, paromomycin (100 μg/mL of final concentration) was added and the cells distributed in 96-well microtiter plates and aliquots of 100 μL. The cells were incubated in a moist box at 30° C. After 2-3 days, resistant clones could be identified. True micronucleus transformants could be distinguished by means of resistance to 6-methylpurine from the macronucleus transformants. During transformation of the macronucleus, about 4 hours after transformation, paromomycin (100 μg/mL final concentration) was added and the cells distributed in 96-well microtiter plates in aliquots of 100 μL. The cells were incubated in a moist box at 30° C. After 2-3 days, resistant clones could be identified. Positive clones were reinoculated in fresh medium with 120 μg/mL paromomycin. By culturing of the cells at this high paromomycin concentration, after a few generations a complete “phenotypic assortment” was reached (Gaertig & Kapler (1999)).

By crossing of the micronucleus transformants with a B*VI strain, homozygous knockout mutants could be produced (Bruns & Cassidy-Hanley, Methods in Cell Biology, Volume 62 (1999) 229-240).

### EXAMPLE 7

### Biolistic Transformation (Microparticle Bombardment)

Transformation of Tetrahymena thermophila occurred by biolistic transformation, as described in Bruns & Cassidy-Hanley (Methods in Cell Biology, Volume 62 (1999) 501-512); Gaertig et al. 91999) Nature Biotech. 17: 462-465) or Cassidy-Hanley et al. (1997 Genetics 146: 135-147)). Handling of the Biolistic® PDS-1000/He Particle Delivery System (BIO-RAD) is described in detail in the corresponding handbook.

6 mg gold particles (0.6 μm; BIO-RAD) were loaded with 10 μg linearized plasmid DNA for transformation (Sanford et al. (199) Biotechniques 3:3-16; Bruns & Cassidy-Hanley (1999) Methods in Cell Biology, Volume 62: 501-512).

Preparation of the gold particles: 60 mg of 0.6 μm gold particles (BIO-RAD) were resuspended in 1 mL ethanol. For this purpose, the particles were mixed 3 times for 1-2 minutes each time on a vortex. The particles were then centrifuged for 1 minute (10,000 g) and the supernatant carefully removed with a pipette. The gold particles were resuspended in 1 mL sterile water and centrifuged as above. This washing step was repeated once, the particles resuspended in 1 mL 50% glycerol and stored at −20° C. in aliquots of 100 μL.

Preparation of transformation: the macrocarrier holder, macrocarrier and stop screens were stored for several hours in 100% ethanol, the rupture disks in isopropanol. A macrocarrier was then inserted into the macrocarrier holder and dried in air.

Loading of the gold particles with DNA: all work occurred at 4° C. Gold particles, prepared vector, 2.5 M CaCl₂, 1 M spermidine, 70% and 100% ethanol were cooled on ice. 10 μL of the linearized vector DNA (1 μg/mL) was added to 100 μL prepared gold particles and carefully vortexed for 10 seconds. 100 μL 2.5 M CaCl₂ was first added, vortexed for 10 seconds and followed by 40 μL 1 M spermidine and vortexed carefully for 10 minutes. After addition of 200 μL 70% ethanol, the particles were vortexed for 1 minute and then centrifuged for 1 minute at 10,000 g. The pellet was resuspended in 20 μL 100% ethanol, centrifuged and then resuspended in 35 μL 100% ethanol.

The particles so prepared were carefully introduced to the center of a macrocarrier with a pipette. The macrocarrier was then stored in a box of hygroscopic silica gel up to transformation.

Transformation: 1 mL of the prepared cells (see above) was introduced into the center of a round filter, moistened with 10 mM tris-HCl (pH 7.5) in a petri dish and inserted into the lowermost insertion strip of the transformation chamber of the Biolistic® PDS-1000/He Particle Delivery System. Transformation occurred with the prepared gold particles at a pressure of 900 psi (two 450 psi rupture disks) and a vacuum of 27 inches Hg in the transformation chamber. The cells were then immediately transferred to an Erlenmeyer flask with 50 mL SPPA medium and incubated at 30° C. without shaking.

### EXAMPLE 8

### Repreparation-Transformation of Tetrahymanol Knockout Mutants with the Genomic Tetrahymanol-Cyclase Plasmid pgTHC

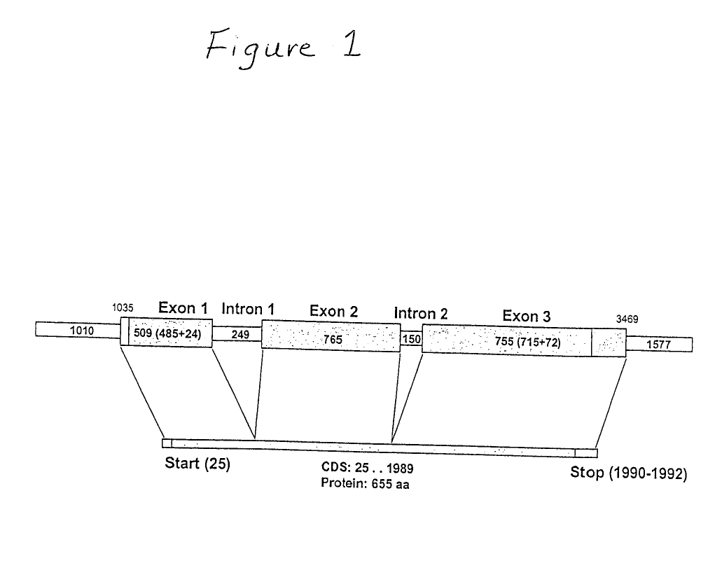

Transformation occurred by analogy to example 4. Tetrahymanol knockout mutants (see example) from Tetrahymena thermophila were used for transformation. Culturing of the cells occurred in SPPA medium with addition of 10 mg/L cholesterol. After transformation (see above) with the genomic fragment of tetrahymanol-cyclase (see FIG. 1), the cells were taken up in SPPA medium without cholesterol and incubated at 30° C. without shaking in an Erlenmeyer flask. After 3 hours, the cells were transferred to 96-well microtiter plates in aliquots of 100 μL. The cells were incubated in a moist, darkened box at 30° C. After about 2-3 days, the first cholesterol autotrophic clones to be identified. After about 5-7 days, the positive clones were reinoculated in fresh medium. By daily reinoculation of the cells over a period of about 2 weeks and repeated isolation of individual cells, a complete “phenotypic assortment” was achieved (Gaertig & Kapler (1999)). These clones were cultured with addition of paromomycin for control in SPPA medium. After about 3-5 days, all the cultures died. The clones had lost their resistance to paromomycin, which is due to loss of the neo-gene by homologous recombination.

### EXAMPLE 9

### Repreparation-Transformation of Delta-6-Desaturase Knockout Mutants with the Delta-6 Plasmid pgDES6

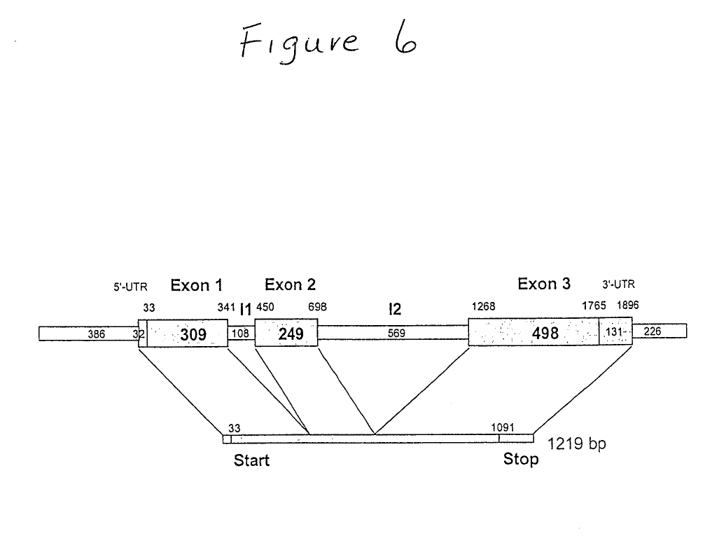

Transformation occurred by analogy to example 4. For transformation, delta-6-desaturased knockout mutants (see example 5) of Tetrahymena thermophila were used. Culturing of the cells occurred in SPPA medium with addition of 200 μg/mL borage oil (20-25% GLA; SIGMA). After transformation (see above) with the genomic delta-6-desaturase fragments (see FIG. 6), the cells were taken up in SPPA medium without borage oil and incubated at 30° C. without shaking in the Erlenmeyer flask. After 3 hours, the cells were transferred in aliquots of 100 μL to 96-well microtiter plates. The cells were incubated in a moist, darkened box at 30° C. After about 2-3 days, the first GLA auxotrophic clones could be identified. After about 5-7 days, the positive clones were reinoculated in fresh medium. By daily reinoculation of the cells over a period of about 2 weeks and repeated isolation of individual cells, a complete “phenotypic assortment” was reached (Gaertig & Kapler (1999)). These clones were cultured for control in SPPA medium with addition of paromomycin. After about 3-5 days, all cultures died. The clones had lost their resistance to paromomycin, which is based on loss of the neo-gene by homologous recombination.

### EXAMPLE 10

### Repreparation-Transformation with the Plasmid pgTHC Coupled with the Neomycin Gene

Transformation occurred by analogy to example 7. A vector constructed as follows was used:

The genomic fragment of tetrahymanol-cyclase (pgTHC) was cleaved with the restriction enzyme Bgl II. The enzyme cleaves the genomic fragment outside of the coding exon in position 4537 in the 3′-untranslated region. After incubation with T4 DNA polymerase to smooth the ends, the neomycin cassette was ligated in the plasmid. For this purpose, plasmid p4T2-1ΔH3 was cleaved with Eco RV/Sma I and the roughly 1.4 kb fragment, containing the neo-cassette, was ligated into the already cleaved plasmid pgTHC into the 3′-untranslated sequence of Tetrahymena triterpenoid-cyclase.

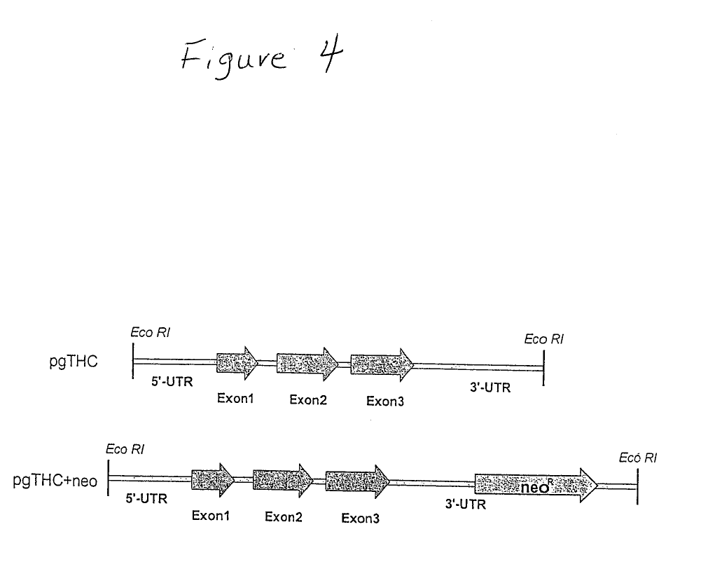

This construct (pgTHC+neo, see FIG. 4) was used for transformation of the tetrahymanol mutants. After transformation (see above), the cells were taken up in SPPA medium without cholesterol and incubated at 30° C. without shaking in the Erlenmeyer flask. After 3 hours, paromomycin was added and the cells transferred in aliquots of 100 μL to 96-well microtiter plates. The cells were incubated in a moist, darkened box at 30° C. After about 2-3 days, the first cholesterol-autotrophic clones could be identified, which were simultaneously resistant to paromomycin. The positive clones were reinoculated in fresh medium without addition of cholesterol or paromomycin. By daily reinoculation of the cells over a period of about 2 weeks and repeated isolation of individual cells, a complete “phenotypic assortment” was achieved (Gaertig & Kapler (1999)). These cells showed good growth even after addition of paromomycin.

### EXAMPLE 11

### Repreparation-Transformation with the Plasmid pgDES6 Coupled with the Neomycin Gene

The transformation occurred by analogy of example 9. A vector constructed as follows was used:

The genomic fragment of delta-6-desaturase (pgDES6) was cleaved with the restriction enzyme Sma BI. This enzyme cleaves the genomic fragment outside of the coding exon at position 747 in the 5′-untranslated region. Plasmid p4T2-1ΔH3 was cleaved with Eco RV/Sma I and the roughly 1.4 kb fragment containing the neo-cassette was ligated in the already cleaved plasmid pgDES6 in the 5′-untranslated sequence of Tetrahymena delta-6-desaturase.

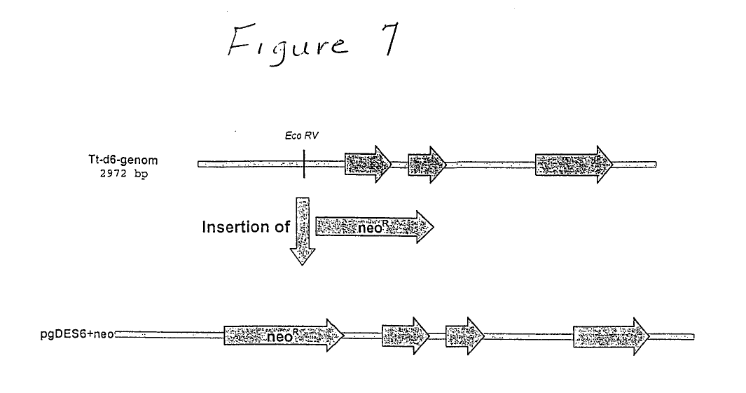

This construct (pgDES6+neo, see FIG. 7) was used for transformation of the delta-6-desaturase mutants. After transformation (see above), the cells were taken up in SPPA medium without borage oil and incubated at 30° C. without shaking in the Erlenmeyer flask. After 3 hours, paromomycin was added and the cells transferred in aliquots of 100 μL to 96-well microtiter plates. The cells were incubated in a moist, darkened box at 30° C. After about 2-3 days, the first cholesterol-autotrophic clones could be identified, which were simultaneously paromomycin resistant. The positive clones were reinoculated in fresh medium without addition of cholesterol or paromomycin. By daily reinoculation of the cells over a period of about 2 weeks and repeated isolation of individual cells, a complete “phenotypic assortment” was achieved (Gaertig & Kapler (1999)). These cells exhibited good growth even after addition of paromomycin.

1. Method for production of recombinant protists, comprising the steps:
a) production of auxotrophic mutant of the protist,
b) transformation of the mutants with recombinant DNA, containing at least one gene for complementation of the corresponding auxotrophy,
c) selection of the recombinant protists on a minimal medium that permits growth only of the corresponding complemented protists.

2. Method according to claim 1, in which production of the auxotrophic mutants occurs by knockout of an essential gene.

3. Method according to claim 2, in which the knocked out gene codes for a triterpenoid-cyclase, a delta-6-desaturase or a delta-9-desaturase.

4. Method according to claim 1, in which the protists are protozoans, preferably ciliates, with particular preference from the genera Paramecium or Tetrahymena, and quite particularly the species Tetrahymena thermophila.

5. Method according to claim 1, in which the recombinant DNA for transformation of the auxotrophic protist mutants contains a gene for a triterpenoid-cyclase, a delta-6-desaturase or a delta-9-desaturase.

6. Recombinant protist, characterized by the fact that it contains a mutation that knocks out an essential gene.

7. Recombinant protist according to claim 6, in which the auxotrophy resulting from the knockout is complemented by transformation of the protist with recombinant DNA.

8. Recombinant protist according to claim 7, in which the recombinant DNA contains a gene for a triterpenoid-cyclase, a delta-6-desaturase or a delta-9-desaturase.

9. Recombinant protist according to claim 6, in which the knocked out gene codes for a triterpenoid-cyclase, a delta-6-desaturase or a delta-9-desaturase.

10. Recombinant protist according to claim 6, in which the protist is a protozoan, preferably a ciliate, with particular preference from the genus Paramecium or the genus Tetrahymena, and quite particularly the species Tetrahymena thermophila.

11. Method for production of recombinant proteins, comprising the steps:
a) production of recombinant protists according to the method of claim 1, in which the recombinant DNA for transformation of the protist additionally contains at least one functional recombinant gene for a protein being expressed,
b) culturing of the recombinant protists and expression of the proteins,
c) isolation of the proteins.

12. Protein production method according to claim 11, in which the recombinant gene for the protein being expressed was isolated from a vertebrate, preferably from a human.


SEQUENCE LISTING (4 sequences)

SEQ ID NO: 1 - DNA, 32 bp, Artificial Sequence
  Primer-bind
ctctttcatacatgcataagatactcataggc

SEQ ID NO: 2 - DNA, 35 bp, Artificial Sequence
  Primer-bind
ggcttggatcctcaaatattttatttttatacagg

SEQ ID NO: 3 - DNA, 30 bp, Artificial Sequence
  Primer-bind
aaaaataaaaaagtttgaaaaaaaaccttc

SEQ ID NO: 4 - DNA, 23 bp, Artificial Sequence
  Primer-bind
gtttagcctgaccgattcagttc